(19) United States
(12) Patent Application Publication
(10) Pub. No.: US 2025/0347682 A1
(43) Pub. Date: Nov. 13, 2025

(54) POPULATION SCREENING SYSTEMS AND METHODS FOR EARLY DETECTION OF CHRONIC DISEASES

(71) Applicant: Regents of the University of Minnesota, Minneapolis, MN (US)

(72) Inventor: Joe Don WEBER, Minnetonka, MN (US)

(21) Appl. No.: 18/869,633

(22) PCT Filed: May 26, 2023
(86) PCT No.: PCT/US2023/023654

§ 371 Date: Nov. 26, 2024

Related U.S. Application Data
(60) Provisional application No. 63/346,713, filed on May 27, 2022.

(51) Int. Cl. G01N 33/52; B01L 3/00; G01N 21/84

(52) U.S. Cl. CPC G01N 33/525; B01L 3/5023; G01N 21/8483; B01L 2200/143; B01L 2300/021; B01L 2300/069; B01L 2300/0825

(57) ABSTRACT

A system for diagnostic screening of a chronic disease includes a test matrix and a user device. The test matrix has a calibration surface including one or more calibration elements and one or more reagents. The user device has image capturing and processing capabilities configured to capture an image and a processor configured to locate, based on the plurality of calibration elements, the test matrix in the image, determine if the test matrix indicates the presence of the chronic disease by comparing a detected contrast of the one or more reagents with the test matrix to an augmented reality trained database, and generate a screening report based on the determination.

### RELATED APPLICATION

This application claims the benefit of U.S. Provisional Application No. 63/346,713 filed May 27, 2022, which is hereby fully incorporated herein by reference.

### TECHNICAL FIELD

The present disclosure relates generally to the field of image processing for early detection of chronic diseases. In particular, the present disclosure relates to systems and methods for providing remote medical screening through image-based analysis of reagents exposed to bodily fluid.

### BACKGROUND

Screening to identify chronic conditions can help lessen the severity of illness or prevent disease by identifying those at risk. Early detection and intervention from screening services can save money and lives. Unfortunately, not everyone who would benefit from screening services receives them, largely due to inadequate access to services. Many individuals and families lack adequate access to healthcare due to cost and location. In some cases, people who need screening services simply do not pursue them due to the difficulty in scheduling a medical appointment amid work and other obligations. Therefore, while clinical tests have been developed to screen for specific conditions, such screening options are often unavailable in non-medical settings.

One area where conventional solutions generally require a clinical visit is the testing of biological materials, such as urinalysis. These tests can include the use of color-based reaction testing, whereby a test pad is exposed to urine, blood, saliva, feces or sweat. Exposure of test pads to biological materials can be used to detect substances associated with chronic conditions before patients are aware that they may have a problem. For example, a urinalysis test can identify traces of protein in a urine sample that can indicate a risk of kidney disease.

Biological material reaction testing, such as urinalysis, is typically performed using dipsticks, which are strips of plastic or paper with a series of reagent test pads thereon. Each reagent test pad on the dipstick is chemically treated with a compound that is known to change color in the presence of particular reactants. The testing process involves exposing the dipsticks to a subject's biological material(s). If the biological material contains quantities of the particular reactants, one or more of the reagent test pads will change color as a result. The magnitude of the change in color is indicative of the amount of the particular reactants that are present. Manual comparison of color shades can be subjective and inaccurate, so many clinicians use specialized electronic readers. While electronic readers provide increased reliability, they are typically highly-calibrated devices that are expensive and not conveniently portable.

### SUMMARY

Thus, there is a need for an accessible and cost effective screening method for early detection of chronic diseases.

The techniques of this disclosure generally relate to systems and methods for screening chronic diseases to promote early detection.

In one aspect, the present disclosure provides for a system for diagnostic screening of a chronic disease. The system includes a test matrix having a calibration surface including one or more calibration elements and one or more reagents and a user device having image capturing and processing capabilities configured to capture an image and a processor. The user device is configured to locate, based on the one or more calibration elements, the test matrix in the image, determine if the test matrix indicates the presence of the chronic disease by comparing a detected contrast of the one or more reagents with the test matrix to an augmented reality trained database, and generate a report based on the determination.

In a second aspect, the present disclosure provides for a kit for performing a diagnostic screen of a chronic disease. The kit includes a test matrix having a calibration surface including one or more calibration elements and one or more reagents and a payment verification code. Entry of the payment verification code via an application on a user device having image capturing and processing capabilities and a processor, causes the user device to capture an image, locate, based on the one or more calibration elements, the test matrix in the image, determine if the test matrix indicates the presence of the chronic disease by comparing a detected contrast of the one or more reagents with the test matrix to an augmented reality trained database, and generate a report based on the determination.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a block diagram of a system for screening chronic diseases according to an embodiment.

FIG. 2 is a flow chart of a method of screening chronic diseases according to an embodiment.

FIG. 3 is an illustration of a test matrix according to an embodiment.

FIG. 4 is a test matrix including a verification code according to an embodiment.

FIG. 5A is a perspective right-side view of a folding sample receptacle in a folded state according to an embodiment.

FIG. 5B is a perspective left-side view of the folding sample receptacle of FIG. 5A.

FIG. 5C is a perspective bottom-up view of the folding sample receptacle of FIG. 5A.

FIG. 6 is a perspective view of the folding sample receptacle of FIG. 5A in a collapsed state.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The present disclosure is directed to systems and methods for processing images captured by a user device to screen for chronic diseases. This is generally accomplished by identifying a test matrix within a captured image and comparing the test matrix to an augmented reality (AR) trained database.

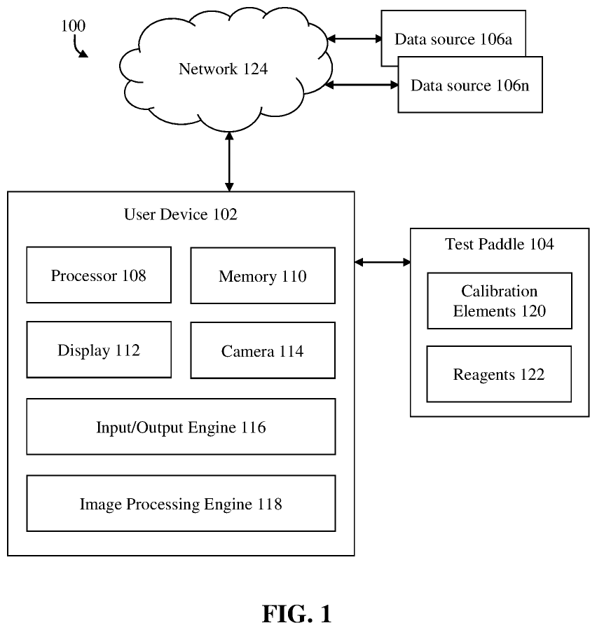

Referring to FIG. 1, a block diagram of a system 100 for screening for chronic disease is depicted, according to an embodiment. System 100 can act as a remote screening system for early detection of chronic diseases and comprises a user device 102, a test matrix 104, and at least one data source 106.

User device 102 generally comprises processor 108, memory 110, display 112, camera 114, and one or more engines, such as input/output engine 116 and image analysis engine 118 as depicted in FIG. 1. Examples of user device 102 include, smartphones, tablets, laptop computers, wearable devices, user equipment (UE), and the like. It is noted that the term “user device” refers to and can be used interchangeably with any of the variety of devices listed above.

Processor 108 can be any programmable device that accepts digital data as input, is configured to process the input according to instructions or algorithms, and provides results as outputs. In an embodiment, processor 108 can be a central processing unit (CPU) configured to carry out the instructions of a computer program. Processor 108 is therefore configured to perform at least basic arithmetical, logical, and input/output operations.

Memory 110 can comprise volatile or non-volatile memory as required by the coupled processor 108 to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, or optical disc storage, for example. The foregoing lists in no way limit the type of memory that can be used, as these embodiments are given only by way of example and are not intended to limit the scope of the present disclosure.

Display 112 is communicatively coupled to input/output engine 116 and is configured to present a graphical user interface (GUI). The GUI can incorporate a dynamic application programming interface (API) that can modify displayed information based upon data recorded by camera 114 or processed by image processing engine 118. For example, a GUI of user device 102 can be dynamically updated based on real time image data processed by image processing engine 118. In embodiments, display 112 can be a touch display or otherwise be configured to directly receive user inputs.

Camera 114 refers to any camera or image sensor capable of providing image capturing and processing capabilities. Examples of camera 114 include digital cameras and phone cameras. Camera 114 can be configured to record and store digital images, digital video streams, data derived from captured images, and data that may be used to construct 3D images. The image data acquired by camera 114 can be communicated to image processing engine 118 for analysis.

Some of the subsystems of system 100, such as input/output engine 116 and image processing engine 118, include various engines or tools, each of which is constructed, programmed, configured, or otherwise adapted, to autonomously carry out a function or set of functions. The term engine as used herein is defined as a real-world device, component, or arrangement of components implemented using hardware, such as by an application specific integrated circuit (ASIC) or field-programmable gate array (FPGA), for example, or as a combination of hardware and software, such as by a microprocessor system and a set of program instructions that adapt the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality: however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

Input/output engine 116 is configured to provide two-way data communication with network 124 via a wired or wireless connection. The specific design and implementation of input/output engine 116 can depend on the communications network(s) over which user device 102 is intended to operate. Input output engine 116 can, via network 124, access stored data from at least one data source 106.

Input/output engine 116 is further configured to receive and process user input, such as that input through display 112. User input received via input/output engine 114 can include at least one of text input, voice commands, tactile input, or the like.

Image processing engine 118 is configured to provide image analysis capabilities for images or video captured by camera 114. In particular, image processing engine 118 is configured to identify the presence of test matrix 104 within a captured image and to then compare the captured image to an AR trained database, such as data source 106. In embodiments, if user device 102 does not include image capture capabilities, such as camera 114, a stored video or image can be communicated to image processing engine 118 for analysis. Although depicted as part of user device 102 in FIG. 1, in some embodiments image processing engine 118 can instead operate on a server or separate device.

Test matrix 104 comprises a calibration surface including one or more calibration elements 120 and one or more reagents 122. Calibration elements 120 enable image processing engine 118 to identify and determine the orientation of test matrix 104 within an image. Calibration elements 120 can incorporate one or more of high contrast, color, or depth elements. In embodiments, calibration elements 120 can correspond to a type of test matrix 104, such as a urinalysis test matrix. Regents 122 are assay reagent squares or reagent pads that can be used to determine an extent of a chemical reaction and the presence of chemicals.

Data source 106 can be a general-purpose database management storage system (DBMS) or relational DBMS as implemented by, for example. Oracle, IBM DB2. Microsoft SQL Server, PostgreSQL, MySQL, SQLite, Linux, or Unix solutions that is trained to interpret AR images corresponding to test matrix 104. Data source 106 can store one or more training data sets configured to facilitate future detection of test matrix 104 within an image. In embodiments, data source 106 can sort training data sets based on calibration elements 120 of each test matrix 104.

In embodiments, artificial intelligence or machine learning models can be implemented to train data source 106. The AI model can be used to estimate the orientation and position of reagents based on calibration elements to speed up the comparison process. The AI model can also be trained to efficiently identify instances where test matrix 104 has not yet been exposed to biological material by recognizing calibration elements are present without any indicated change to reagents 122. The trained machine learning model can also be used for identifying differences in pixel intensity based on differences in illumination conditions of a captured image to training data. In an embodiment, training data can include a plurality of image data having test matrixes in different positions within the image data and an indication of whether each test matrix indicates presence of a particular chronic disease.

In embodiments, unreacted reagents 122 are configured to be the same color as the calibration surface of test matrix 104 to simplify analysis of test results. In such embodiments, the presence of test matrix 104 can be determined entirely though calibration elements 120 and any color variances across test matrix 104 can be assumed to result from a chemical reaction based on the expectation that the test matrix 104 would otherwise be uniform. In such embodiments, calibration elements can be analyzed to ensure that no objects are partially obscuring test matrix 104 in the image.

Another aspect of the present disclosure is a home testing kit. Such a kit can comprise a test matrix, such as test matrix 104, to enable screening for chronic diseases to be accomplished at home or in any other environment, a folding sample receptacle, and a verification code to verify the test kit is legitimately purchased by the user.

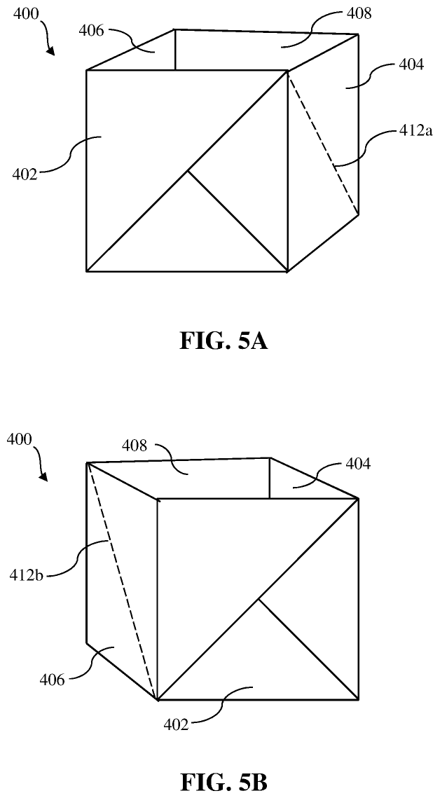

In embodiments, the folding receptacle can be packaged as a flat sheet that includes instructions for folding into a fluid-tight, open-faced box as shown in FIGS. 5A-5C and described in detail later. The instructions can include one or more of colored portions, line indications, perforations, or indentations on the flat sheet or can be one or more pages of instructions external to the flat sheet. In embodiments, the one or more pages of instructions can include instructions for use of the test matrix and/or verification code.

The verification code can be sent to a user device after purchase of the kit or can be physically included within the kit. Instructions for use also can be included in the kit. The verification code can also be configured to provide the AR trained database with preliminary information about the test matrix to improve detection of the test matrix within subsequently captured images. Such previous use information may include dimensions of the test matrix and locations of calibration elements and reagents on the test matrix. In some embodiments, verification code can be used to determine the time since the home testing kit was purchased to provide an estimate of whether the test matrix is still reliable.

Home testing kits can optionally also include one or more of a blot pad for removing excess urine form test matrix, a container configured to store a urine sample, and a calibration placemat for positioning the test matrix during image capture. The blot pad can prevent image distortion that may occur from excess urine distorting calibration elements or reagents. The container can be a collapsible cup, test tube, or any structure capable of holding a fluid. The calibration placemat can include orientation instructions for image capture, one or more additional calibration elements, and a designated region to place an exposed test matrix.

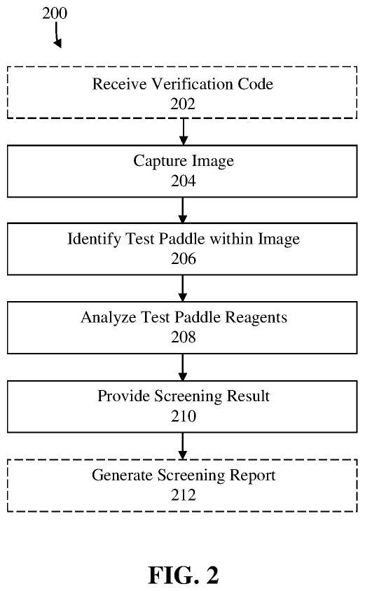

Referring to FIG. 2, a flow chart of a method 200 for providing screening results using a system 100 is depicted according to embodiments. Optionally, at 202, a verification code is received by a user device. The verification code can verify purchase of an associated test matrix. At 204, an image is captured by the user device or is otherwise communicated to an image processing engine that can be internal or external to the user device. A prompt or alert can be sent to the user device to request access to the image capture capabilities of the user device.

At 206, the test matrix is identified within the image based on the presence of calibration elements and comparison to an AR trained database. Incorporation of a calibration placemat can simplify test matrix detection. An AI model can be trained using training examples to identify calibration elements in images, and the trained AI model may be used to analyze the captured image and identify the reagents. In some embodiments, an object detection algorithm may be used to detect the test matrix in the captured image, and the reagents can then be identified based on their positions relative to the calibration elements.

At 208, the reagents of the test matrix are evaluated to determine the presence of chemicals of interest. In embodiments, the reagents can be organized in asymmetric shapes or designs to for quicker detection of each respective reagent. In embodiments where each reagent is the same color as the surrounding test matrix surface, any change in the color of the reagent can efficiently indicate the presence of a chemical. Because of this, color or pixel intensity analysis may not be necessary to provide accurate screening results.

In some embodiments, the type of analysis performed on the received image may depend on information contained in the verification code or detected calibration elements. For example, if the received verification code is indicative of a particular urinalysis test matrix, the AR trained database can limit the analysis of test matrix reagents to only the training sets incorporating the associated urinalysis test matrix.

At 210, screening results are provided to the user. Optionally, at 212, a report of the screening results can be prepared, including information designed to assist the user in understanding the screening results or to direct the user to appropriate health care providers. In some embodiments, the user can save or otherwise forward their screening report for future reference.

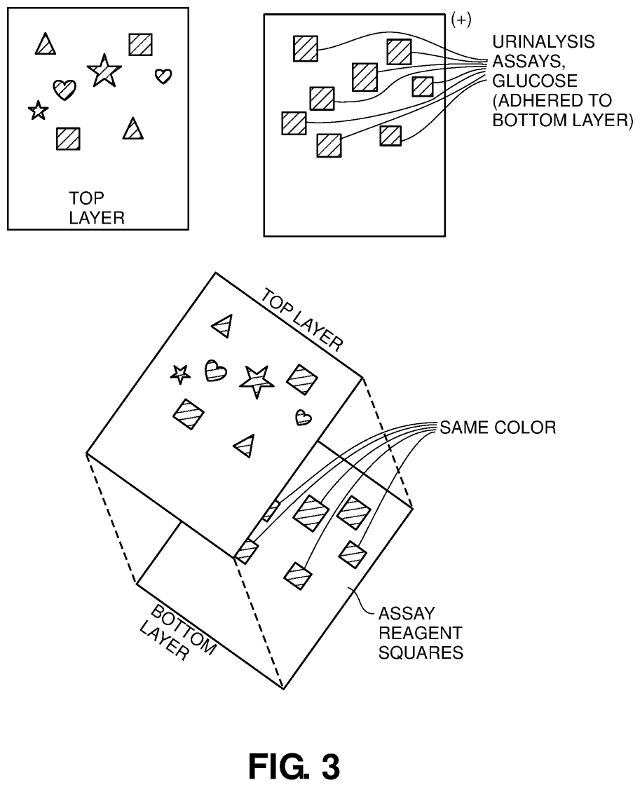

Referring now to FIG. 3, a two-layer test matrix design is depicted according to embodiments. The same color can be used for reagents and the top layer of the test matrix to enable efficient detection glucose and other substances. In such embodiments, a separate calibration element (not shown) can be present on the test matrix so that the test matrix can be identified even in an unused or unchanged state.

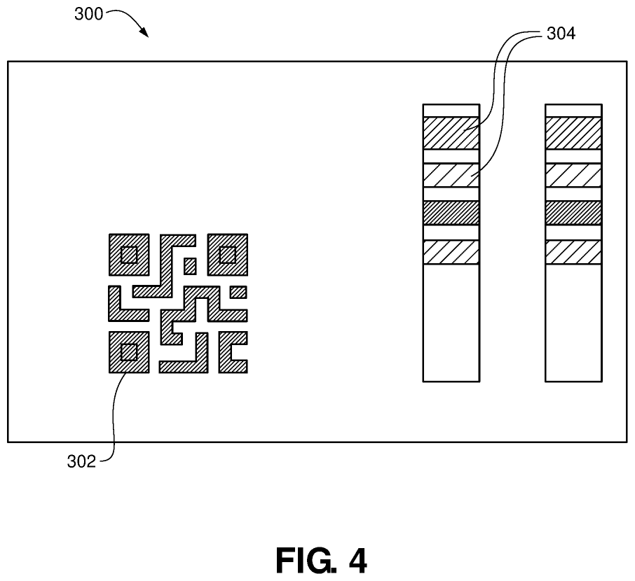

Referring to FIG. 4, a test matrix 300 is depicted according to embodiments. Test matrix 300 can comprise calibration element 302 and reagents 304. In embodiments, calibration element 302 can also serve as a verification code.

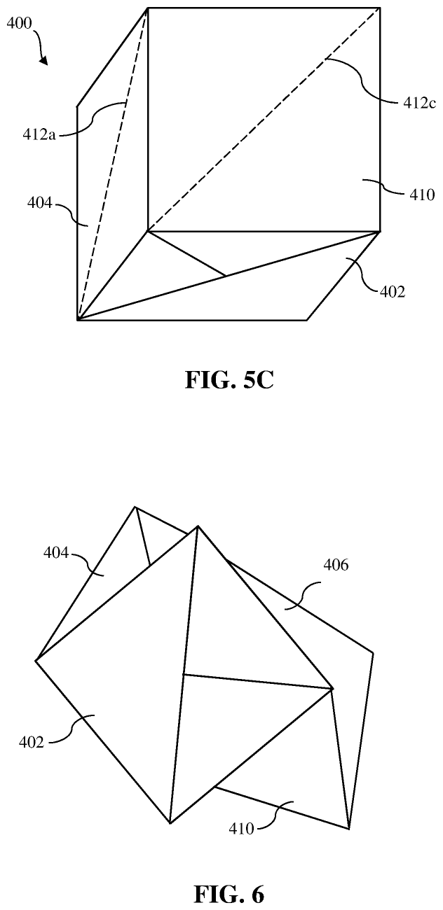

Referring to FIGS. 5A-5C foldable sample receptacle 400 is depicted according to embodiment. When in a folded state, foldable sample receptacle 400 includes front panel 402, first side panel 404 having inward fold 412a, second side panel 406 having inward fold 412b, back panel 408, and bottom panel 410 having inward fold 412c. When in the folded state, foldable sample receptacle 400 is water-tight and can simplify the test matrix dipping process by providing a disposable receptacle for urine that can be efficiently packaged as a flat sheet. Inward folds 412a-c improve the stability of foldable sample receptacle 400 and allow for easier user assembly. By applying pressure to the exterior of the panels with inward folds 412a-c, foldable sample receptacle 400 can be stored in a mostly flat state as shown in FIG. 6.

In embodiments, foldable sample receptacle 400 can comprise the test matrix such that the test matrix appears on the inside surface of bottom panel 410 when foldable sample receptacle 400 is in a folded state. In other embodiments, test matrix can be sized to be placed inside foldable sample receptacle 400 such that the user can see the results of the test matrix without removing it from foldable sample receptacle 400 Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A system for diagnostic screening of a chronic disease, comprising:
a test matrix having a calibration surface including a one or more calibration elements and one or more reagents;
a user device having image capturing and processing capabilities configured to capture an image and a processor configured to:
locate, based on the one or more calibration elements, the test matrix in the image;
determine if the test matrix indicates the presence of the chronic disease by comparing a detected contrast of the one or more reagents with the test matrix to an augmented reality trained database; and
generate a report based on the determination.

2. A kit for performing a diagnostic screen of a chronic disease, comprising:
a test matrix having a calibration surface including one or more calibration elements and one or more reagents; and
a verification code, wherein entry of the verification code via an application on a user device having image capturing and processing capabilities and a processor, causes the user device to:
capture an image;
locate, based on the one or more calibration elements, the test matrix in the image;
determine if the test matrix indicates the presence of the chronic disease by comparing a detected contrast of the one or more reagents with the test matrix to an augmented reality trained database; and
generate a report based on the determination.
